(19) United States
(12) Patent Application Publication
(10) Pub. No.: US 2023/0118853 A1
(43) Pub. Date: Apr. 20, 2023

(54) PAYER-PROVIDER COLLABORATION

(71) Applicant: KAID Health, Inc., Boston, MA (US)

(72) Inventor: Kevin Agatstein, Newton, MA (US)

(21) Appl. No.: 17/502,437

(22) Filed: Oct. 15, 2021

(51) Int. Cl. G16H 20/00; G06Q 40/08; G16H 15/00

(52) U.S. Cl. CPC G16H 20/00; G06Q 40/08; G16H 15/00

(57) ABSTRACT

A payer-provider collaboration system receives data from healthcare providers and claims data provided by health insurers to create a common data model, receives business rules and definitions of opportunities of interest from the health insurers, and processes data from the common data model and the business rules and definitions to identify care improvement opportunities to address. A user interface presents care improvement opportunities to healthcare providers, and present to the health insurers in a second user interface on a display device, data about opportunities addressed by the healthcare providers, and data identifying incentive payments to pay to the healthcare providers based on the data about opportunities addressed by the healthcare providers.

### BACKGROUND

The subject matter of the present disclosure relates generally to providing a system for healthcare collaboration between healthcare providers and health insurers.

The delivery of high-quality, coordinated, appropriate reimbursed healthcare today suffers from inefficiencies and a lack of integration between healthcare providers and health insurers. Uncoordinated care management contributes significantly to healthcare costs. Care management involves activities that address coordination of care, waste, and the management of health conditions. Unfortunately, today most care management programs are operating at arm's length, both physically and informatically, from the actual treating providers. This is true whether the care managers are employed by the payer or by the health system. Thus, care remains under-coordinated, and patients remain noncompliant. Further, because good care management is expensive, only a small fraction of patients get the service.

Bridging this gap between care provision and care management requires new data and tools. Specifically, care management teams need access to the patient's medical notes as well as analytics to automatically find actionable insights in this treasure trove of information. Fortunately, the technology to do so, Natural Language Understanding (NLU) and Artificial Intelligence (AI), are emerging. Additionally, they need technology to ensure the identified patient interventions are performed in timely basis, including the distribution of any applicable financial incentives for doing so.

Thus, there is a need for a platform to ensure coordination between health insurers and healthcare provides so that each patient's care is informed, coordinated, and fully coded & documented.

### SUMMARY

An aspect of the present disclosure involves a system and method to providing a system for healthcare collaboration between healthcare providers and health insurers.

A payer-provider collaboration system includes a memory storing computer-readable instructions, and a processor, coupled to the memory, configured to execute the computer-readable instructions to receive data from one or more healthcare providers and claims data provided by one or more health insurers to create a common data model, receive business rules and definitions of opportunities of interest from one or more health insurers, process data from the common data model and the business rules and definitions of opportunities of interest to identify one or more care improvement opportunities to address, and present, in a first user interface on a display device, the one or more care improvement opportunities to the one or more healthcare providers, and present to the one or more health insurers in a second user interface on a display device, data about opportunities addressed by the one or more healthcare providers, and data identifying incentive payments to pay to the one or more healthcare providers based on the data about opportunities addressed by the one or more healthcare providers.

The one or more care improvement opportunities identify actions to take to improve care and drive completion of intervention across the one or more healthcare providers.

The common data model is created by the processor, wherein the processor is further configured to receive structured and unstructured data from the one or more healthcare providers, process the unstructured data through a natural language processing (NLP) pipeline to create additional structured data, receive the claims data from one or more health plan claims systems of the one or more health insurers, and create the common data model using the structured data, the additional structured data, and the claims data from the one or more health plan claims systems.

The one or more care improvement opportunities are presented in an electronic medical record (EMR).

The business rules and definitions of opportunities of interest include clinical definitions, and priorities and healthcare incentives for addressing management of care opportunities by the one or more healthcare providers.

The processor is further configured to present a third interface, to the one or more healthcare providers, to present the one or more care improvement opportunities, and to document one or more actions taken to address the one or more care improvement opportunities.

The processor is further configured to present, to the one or more health insurers a fourth user interface for executing searches to determine one or more care improvement opportunities; and a fifth user interface to identify, from the determined one or more care improvement opportunities, the one or more actions taken by the one or more healthcare providers regarding the determined one or more care improvement opportunities, to verify the one or more actions taken regarding the determined one or more care improvement opportunities, and to execute payment of incentives to the one or more healthcare providers in response to the verification of the one or more actions taken regarding the determined one or more care improvement opportunities, wherein the incentives are tied to database for translation to monetary compensation values and rules.

The processor is further configured to determine the one or more care improvement opportunities by analyzing the medical records, wherein the one or more care improvement opportunities include coding actions, quality and care improvement interventions, revenue enhancing interventions based on a review of patient medical records and at least one patient profile,

The at least one patient profile includes at least a patient name, data regarding drug exposures, and health condition occurrences.

The one or more care improvement opportunities to address include one or more of identification of gaps in healthcare actions and identification of additional services to provide a patient, identification of gaps in coding and documentation of the one or more care improvement opportunities, identification of social determinants of health (SDoH) and activities of daily living (ADL) limitations to mitigate, identification of interventions for care not being implemented, identification of failure to follow through with select elements of a treatment plan, and identification of an impact of the one or more care improvement opportunities on operational, clinical, and financial metrics.

The claims data provided by one or more health insurers include member identifiers, date of service, medical procedure and service codes, diagnosis coded, point of service code, and other medical claim elements.

The processor is further configured to processes the data from the common data model and the business rules and definitions of opportunities of interest to identify the one or more care improvement opportunities to address by performing analytics on the common data model and the business rules and definitions of opportunities of interest.

The data from one or more healthcare providers and from claims data provided by one or more health insurers are provided to create the common data model using standardized data exchange protocols that define inputs and outputs of interoperable clinical data associated with patients, clinicians, and automated data feeds.

The processor is further configured to identify the one or more care improvement opportunities to address by matching a patient identifier from the one or more healthcare providers with a patient member identifier from the one or more health insurers.

The processor is further configured to present, in the at least one of the first user interface and the second user interface, the care improvement opportunities including a health issue to address, a priority scores based on a clinical impact score, a financial impact score, an ease-to-address score, a target time frame for the one or more healthcare providers to address the health issue, identification of incentive associated with timely and successful completion of the corrective action, and rules controlling target distribution of the incentives among all members of the one or more healthcare providers that address the issue.

The processor is further configured to filter the one or more care improvement opportunities by type, urgency, priority based on clinical priority, financial priority, and likely ease to address, and eligible incentives, and to push the one or more care improvement opportunities to a care team associated with each patient in a target cohort.

The common data model is configured to provide physical, network, and process security measures for protected health information.

### BRIEF SUMMARY OF THE DRAWINGS

The accompanying drawings, which are incorporated in and form a part of the specification, illustrate examples of the subject matter of the present disclosure and, together with the description, serve to explain the principles of the present disclosure. In the drawings:

FIG. 1 illustrates a system 100 for providing payer-provider collaboration.

FIG. 2 illustrates a flow diagram for providing payer-provider collaboration.

FIG. 3 illustrate processing of data to generate the common data model.

FIG. 4 illustrates a natural language processing (NLP) pipeline.

FIG. 5 is a device used in the payer-provider collaboration system.

FIG. 6 is a flow chart of a method 00 for providing a system for healthcare collaboration between healthcare providers and health insurers.

### DETAILED DESCRIPTION

The following detailed description is made with reference to the accompanying drawings and is provided to assist in a comprehensive understanding of various example embodiments of the present disclosure. The following description includes various details to assist in that understanding, but these are to be regarded merely as examples and not for the purpose of limiting the present disclosure as defined by the appended claims and their equivalents. The words and phrases used in the following description are merely used to enable a clear and consistent understanding of the present disclosure. In addition, descriptions of well-known structures, functions, and configurations may have been omitted for clarity and conciseness.

Aspects of the present disclosure are directed to providing a system for healthcare collaboration between healthcare providers and health insurers.

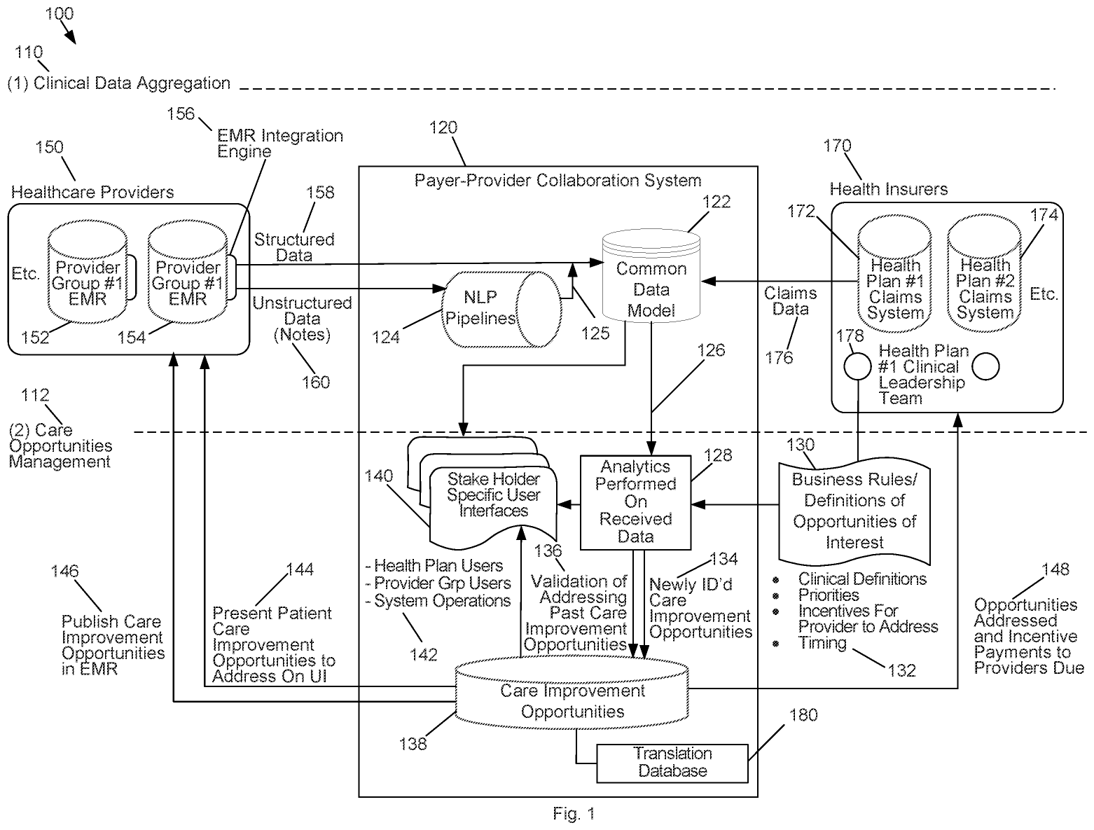

FIG. 1 illustrates a system 100 for providing payer-provider collaboration 100.

In FIG. 1, the payer-provider collaboration system 100 includes a first section providing clinical data aggregation 110 and a second section providing care opportunities management 112. A payer-provider collaboration system 120 is coupled to healthcare providers 150 and to health insurers 170. The healthcare providers 150 may include one or more provider groups that create, maintain, and modify electronic medical records (EMR) 152, 154. An electronic health record (EHR), or electronic medical record (EMR), refers to the systematized collection of patient and population electronically-stored health information in a digital format. These records can be shared across different health care settings. Records are shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information. An EMR integration engine 156 enables the flow of healthcare data, such as patients' personal information, allergies, medications, diagnosis history, radiology test results, etc., across disparate clinical data systems, and provides seamlessly sharing patient healthcare data from one electronic system to another, such as patient demographics, medical history, medications, allergies, lab results, radiology results, etc. The EMR integration engine 156 facilitates improvements to workflows, optimizes the delivery of care, and supports the exchange of clinical, financial, and operational data. The EMR integration engine 156 provides a comprehensive view of patients to clinicians, medical professionals, and businesses that sell directly to healthcare delivery organizations. Structured data 158 and unstructured data 160 is provided to the payer-provider collaboration system 120 from the healthcare providers 150.

Health insurers 170 may include one or more health plan claims systems 172, 174. The health plan claims systems 172, 174 provide claims data 176 to the payer-provider collaboration system 120. One or more health plan clinical leadership teams 178 create and modify as necessary business rules and definitions of care improvement opportunities 130, which are also provided to the payer-provider collaboration system 120. These may include clinical definitions, priorities, incentives for providers to address, and timing parameters 132. Clinical/business rules 130 are reconciled with patient clinical states from the provider group EMRs 152, 154 to identify interventions for gaps to address.

The payer-provider collaboration system 120 may include one or more natural language processing (NLP) pipelines 124 that receive the unstructured data 160 from healthcare providers 150. NLP pipelines 124 extract text from documents via optical character recognition (OCR). the extracted text is then passed through a number of different NLP models, including: text pre-processor, a tokenizer, a tagger to obtain parts of speech, and a parser to split the text into sentences. The processed text is then processed through a Clinical Named Entity Recognition Model (NER) to predict entities, an information extraction model to get information about entities, e.g., “negation”, an entity linking model that links entities to CUIs, and relationship extraction to relate entities to each other.

NLP pipeline 124 processes conditions, medications, laboratory work and testing, procedures, social determinants of health (SDOH), knowledge gaps, treatment plan, health behaviors, and client defined actions. The one or more NLP pipelines 124 process the unstructured data 160 to generate additional structured data 125. The structured data 158 the additional structured data 125 from the healthcare providers 150, and the claims data 176 from the health insurers 170 are used to create a common data model 122. The common data model 122 may use standardized data exchange protocols that define inputs and outputs of interoperable clinical data associated with patients, clinicians, and automated data feeds.

Claims data 176 from health insurers 170 can be provided in a number of different formats, typically a flat file with member ID, date of service, current procedural terminology (CPT) code, diagnosis code, point of service code, and other elements typical of a medical claim. The claims data 176 provided by one or more health insurers 170 may include member identifiers, date of service, medical procedure and service codes, diagnosis coded, point of service code, and other medical claim elements.

Data in the common data model 122 may be searched to identify patients/encounters of interest, and/or to view patient clinical summaries. Analytics 128 are used to process data 126 from the common data model 122 and the business rules and definitions of opportunities of interest 130 to identify one or more care improvement opportunities to address 134. Analytics 128 may involve finding patients who satisfy the clinical criteria for care improvement opportunities 134 defined by the health plans 172, 174. The common data model 122 may further include physical, network, and process security measures to provide protected health information. The identification of the one or more care improvement opportunities to address 134 is made by matching a patient identifier from the one or more healthcare providers 150 with a patient member identifier from the one or more health insurers 170.

The care improvement opportunities 134 involve actions for the healthcare providers to address. For example, the care improvement opportunities 134 may include gaps in hierarchical condition category (HCC) coding and documentation, e.g., diabetics with unassociated renal disease or immunosuppression, patients with documented depression without a corresponding diagnosis code, healthcare effectiveness data and information set (HEDIS)/quality measures, e.g., finding patients self-paying for indicated statins, patients in need of additional interventions, e.g., patients with documented suicidal ideation not being treated, patients with consistently high blood pressure not being treated, patients not following through with select elements of their treatment plan, e.g., referrals to specialists that did not occur, ordered testing that was never performed, and patients with significant, unaddressed SoDHs or limitations in activities in daily living (ADL), e.g., various non-clinical issues that require intervention, such as food insecurity, job loss, transportation issues, and communication barriers. The care improvement opportunities 134 enable the impact of the above on key operational, clinical, and financial metrics to be measured.

Patient care improvement opportunities to address 134 are generated using a predetermined data set. Each patient care improvement opportunity in the database 138 includes a patient identity, e.g., Jane Smith, patient identifiers, e.g., [health insurance ID, medical record number, etc., an opportunity name that provides a textual description of what the health plan is looking to address, e.g., assess a known diabetic with potentially depressed kidney function for nephropathy, a patient source defining how the patient got assigned this opportunity, e.g., through manual user entry, uploaded list (CSV file), or most commonly a “cohort” created from searching the common data model 122. The database 138 may further include a target completion date, incentives points for timely completion, priority scores, e.g., three numbers, each 0-10, representing a financial impact score, clinical impact score, an ease-to-address score, target incentives distribution (3 percentages that sum to 100%, representing share of incentives to physician, clinical staff, and administrative staff), associated content, and a status of opportunity.

Stakeholder specific user interfaces 140 may be generated based on data from the common data model 122, and results from the analytics 128. Stakeholder specific user interfaces 140 are used by authenticated health plan users, provider users, and operational staff to interact with the data, and act on identified opportunities. The stakeholder specific user interfaces 140 may include health plan user interfaces, provider group user interfaces, and system operator interfaces 142.

The analytics 128 may identify new care improvement opportunities 134 that are maintain in a care improvement opportunities database 138. Care improvement opportunities 138 may include a health issue to address, priority scores based on a clinical impact score, a financial impact score an ease-to-address score, a target time frame for the one or more healthcare providers to address the health issue, and an incentive associated with timely and successful completion of the corrective action, and rules controlling target distribution of the incentives among all members of the one or more healthcare providers that address the issue. Care improvement opportunities 138 may further include coding actions, quality and care improvement interventions, and revenue enhancing interventions based on a review of patient medical records and one or more patient profiles. A patient profile may include a patient name, data regarding drug exposures, and health condition occurrences. Care improvement opportunities 138 identify actions to take to improve care and drive completion of intervention across the one or more healthcare providers 150. The validation of past care improvement opportunities 136 may be maintained in the care improvement opportunities database 138. These may be validated through the stakeholder specific user interfaces 140. Care improvement opportunities to address 144 include one or more of identification of gaps in healthcare actions and identification of additional services to provide a patient, identification of gaps in coding and documentation of the one or more care improvement opportunities, identification of social determinants of health (SDoH) and activities of daily living (ADL) limitations to mitigate, identification of interventions for care not being implemented, identification of failure to follow through with select elements of a treatment plan, and identification of an impact of the one or more care improvement opportunities on operational, clinical, and financial metrics.

Patient care improvement opportunities to address 144 may be presented from the care improvement opportunities database 138 to the healthcare providers 150. For example, the one or more care improvement opportunities 144 from the care improvement opportunities database 138 may be presented in a first of the stakeholder specific user interfaces 140 on a display device to the one or more healthcare providers150. A filter control may be provided in the first of the stakeholder specific user interfaces 140 to filter the care improvement opportunities by type, urgency, priority based on clinical priority, financial priority, and likely ease to address, and eligible incentives, and to push the one or more care improvement opportunities to a care team associated with each patient in a target cohort.

Additional interfaces may be presented to the healthcare providers 150 or health insurers 170. For example, a third interface at the one or more healthcare providers 150 to present the one or more of the care improvement opportunities 138 and to document one or more actions taken to address the one or more care improvement opportunities 138. Care improvement opportunities may also be published 146 in one or more of the EMRs 152, 154.

The care improvement opportunities database 138 may also identify to the health insurers 170 incentive information 148 that includes improvement opportunities that were address and incentive payments that are due to the healthcare providers 170. For example, data about opportunities addressed by the one or more healthcare providers and identifying incentive payments to pay to the one or more healthcare providers 148 may be presented in a second of the stakeholder specific user interfaces 140 on a display device to the health insurers 170. A fourth user interface may be used to presented to the one or more health insurers 170 to use for executing searches to determine one or more care improvement opportunities 138. A fifth user interface may be used to identify one or more actions taken by the healthcare providers 150 regarding the care improvement opportunities 138, to verify the one or more actions taken regarding the care improvement opportunities 138, and to execute payment of incentives to the healthcare providers 150 in response to the verification of the one or more actions taken regarding the care improvement opportunities 138, wherein the incentives are tied to a translation database 180 for translation of the satisfied incentives to monetary compensation values and rules.

Thus, payer-provider collaboration system 120 leverages the entirety of the medical record, not just the structured EMR data 152, 154 and claim data 176, but also the medical documentation to identify actionable activities the care team should consider. The payer-provider collaboration system 120 ensures that healthcare providers 150 are able to better manage their patients as well as align with other providers in the delivery of coordinated care. The payer-provider collaboration system 120 continually identifies opportunities to intervene to improve care, drives completion of the intervention across the organization, and ensures healthcare providers are incentivized for their activities. The payer-provider collaboration system 120 combines EMR, analytics, workflow automation, and incentive management into a single platform to drive informed, collaborative care. The payer-provider collaboration system 120 promotes and incentivizes actionable campaigns from the health insurers 170 to healthcare providers 150, identifies care improvement opportunities 138 directly from the EMRs 152, 154 of healthcare providers 150 to drive reimbursement and quality. The payer-provider collaboration system 120 generates new revenues for the healthcare providers 150 to address clinical and operational issues, while better addressing patient's needs.

A first of the stakeholder specific user interfaces 140 may be used to address a systemic issue. A health insurer 170 becomes aware of a systemic failure in the delivery of key elements of care and/or a failure to fully document clinical encounters. A patient ID from a healthcare provider 150 is matched in the payer-provider collaboration system 120 with the patient's corresponding member ID in a health plan claims system 172, 174. A first of the stakeholder specific user interfaces 140 may be used to define a systemic issue to address, including: (i) naming the health issue to address, (ii) assigning it three “priority” scores, clinical impact, financial impact, and ease-to-address, (iii) a target time frame for each clinician to address the issue, (iv) incentive points associated with timely and successful completion of the corrective action, and (v) target distribution of the incentives points among all members of a patient's care team that address the issue. Once finalized in the health plan claims system 172, 174, a set of patients, i.e., a cohort to which the previously identified care improvement opportunity applies, is identified. The payer-provider collaboration system 120 than pushes the care improvement opportunity to a care team of the healthcare providers that is associated with each patient in the target cohort.

A member of the care team of one or more of the healthcare providers 150 determines care improvement opportunities 144 to be addressed on the patient one or more of the healthcare providers 150 are responsible for. A care team member is able to filter the care improvement opportunities 144 by type, urgency, by priority (clinical priority, financial priority, likely ease to address), and eligible incentives. Upon seeing such a care improvement opportunity on a patient, the care team member reviews it, and may choose to take action to address. Upon successfully completing the task, the user marks the issue as resolved. Alternatively, the user may ignore the task, or close it unresolved and provide a reason. If the issue is successfully resolved in a timely manner, the provider is eligible for any incentive associated with that opportunity.

A queue of care improvement opportunities that have been marked as closed is automatically created for the health insurers for validation 138. Health insurers 170 can validate any task in two ways. First, they can review it outside the payer-provider collaboration system 120 and mark it closed. Second, they can query the common data model 122 to identify data that suggests the task is closed. The health insurer 170 can then choose to take action on closed opportunities that cannot be verified.

A key to effectuating the flow described above is the ability of the health insurers 170 to use a user interface to generate a list of patients for whom they want a specific action taken, i.e., referred to a cohort. All data in the common data model 122 may be used to construct a query to create a cohort, including diagnoses, medication exposure, testing value, etc. Further, cohort definitions can include whether a patient is already in another cohort, e.g., do not add patient X to March's “woman over age 45 without a recent mammogram” cohort if the patient was in February's “woman over age 45 without a recent mammogram” cohort.

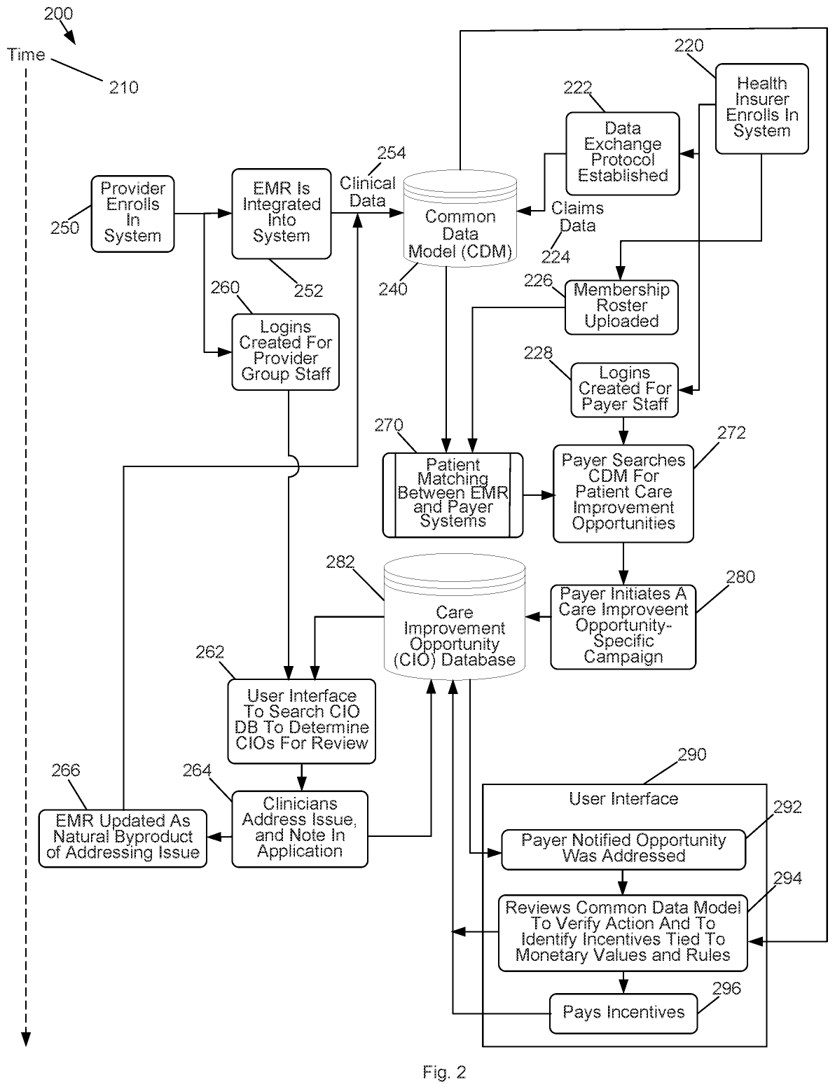

FIG. 2 illustrates a flow diagram 200 for providing payer-provider collaboration.

In FIG. 2, the flow diagram 200 is shown against a timeline 210. A health insurer enrolls in the payer-provider collaboration system 220. A data exchange protocol 222 is established for providing claim data 224 to common data model 240. On the other side, a provider enrolls in the payer-provider collaboration system 250. EMR data is integrated into the payer-provider collaboration system 252 to provide clinical data 254 to the common data model 250. After the health insurer enrolls in the payer-provider collaboration system 220. a membership roster is uploaded 226. Logins are created for the payer staff 228. On the healthcare provider side, logins are created for the healthcare provider group staff 260. The membership roster data is provided for patient matching between EMR data and data from the payer systems 270. The logins created or the payer staff 228 and data reflecting the patient matching between EMR data and data from the payer systems 270 are provided for performing payer searches the common data model 240 for patient care improvement opportunities 272. The payer initiates a care improvement opportunity specific campaign 280. Care improvement opportunities are identified and provided to the care improvement opportunity (CIO) database 282.

On the healthcare provider side, a user interface 262 is provided for receiving logins created for the provider group staff 260 and to perform a search of the care improvement opportunity database 282 for new care improvement opportunities. Clinicians address issues associated with the care improvement opportunities and note actions in an application 264. The issues associated with the care improvement opportunities and actions noted in an application 264 are provided to the care improvement opportunity database 282. The EMR data is also updated as a natural byproduct of addressing any of the issues 266. The EMR data is also provided back to the common data model 240.

A notification is provided in user interface 290 notifying a payer of a care improvement opportunities from the care improvement opportunities database 282. From the user interface 290, the payer is notified that a care improvement opportunity was addressed 293, the common data model is reviewed to verify action and to identify incentives tied to monetary values and rules 294. After the common data model is reviewed to verify action and to identify incentives tied to monetary values and rules 294, pay incentives are provided 296 to improvement opportunities database 282. Verification of action and identification of incentives that are tied to the monetary values and rules are provided to the care improvement opportunities database 282.

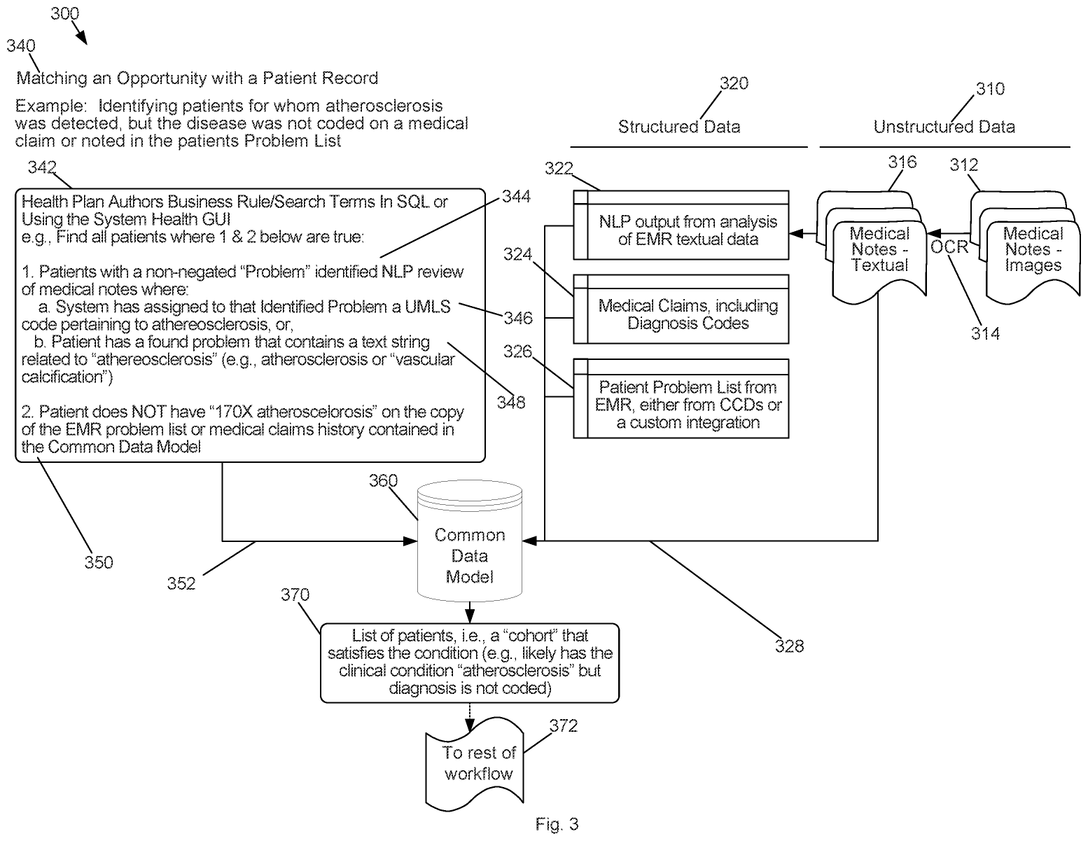

FIG. 3 illustrate processing of data to generate the common data model 300.

In FIG. 3, unstructured data 310 and structured data 320 are shown being processed. The unstructured data 310 includes medical notes and images 312 being scanned by optical character recognition (OCR) 314 to produce textual medical notes 316. NLP output from analysis of EMR textual data 322 is generated using the textual medical notes 316 and provided to the common data model 360. Medical claims data 324 is also provided to the common data model 360. The medical claims data may further include diagnosis codes. Patient problem lists from the EMR data is provided to the common data model 360. The patient problem list 326 may be from Continuity of Care Documents (CCDs) or from a custom integration.

A care improvement opportunity is matched with a patient record 340. For example, patients for whom atherosclerosis was detected may be identified, but the disease may not be coded on a medical claim or noted in the patients problem list. Health plan authors business rule/search terms in SQL or use the system health GUI 342, e.g., find all patients where 1 and 2 conditions below are true. The first condition 344 involves patients with a non-negated problem identified NLP review of medical notes. For the first condition 344, a system has assigned to that identified problem a UMLS code pertaining to atherosclerosis 346, or a patient has a found a problem that contains a text string related to atherosclerosis 348, e.g., atherosclerosis or vascular calcification. The second condition involves a patient that does not have 170× atherosclerosis on the copy of the EMR problem list or medical claims history contained in the common data model 350. The health plan business rules/search results 352 are provided to the common data model 360. The textual medical notes 316 are also provided 340 to the common data model 360.

A list of patients that satisfies both the first condition 344 and the second condition 350 is provided by the common data model 360. For example, the list of patients that satisfies both the first condition 344 and the second condition 350 is referred to as a “cohort”, e.g., likely has the clinical condition “atherosclerosis” but diagnosis is not coded 370. The process then continues to the rest of the workflow 372.

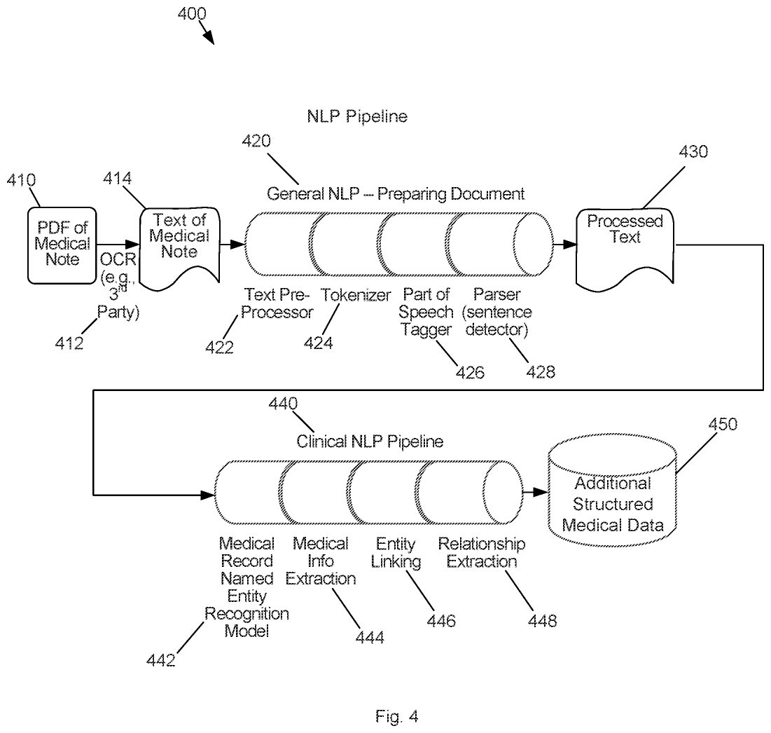

FIG. 4 illustrates a natural language processing (NLP) pipeline 400.

In FIG. 4, a general NLP pipeline 420 and a clinical NLP pipeline 440 are illustrated. An image of a medical notes, such as a portable document formatted (PDF) document, is provided 410. The PDF document 410 is scanned and processed using optical character recognition (OCR) 412. The text of the medical note is generated 414 and provided to the general NLP pipeline 420. The general NLP pipeline 420 includes a text pre-processor 422, a tokenizer 424, a part of speech tagger 426, and a parser (sentence detector) 428. Processed text 430 is produced and provided to the clinical NLP pipeline 440. The clinical NLP pipeline 440 includes a medical record named entity recognition model 442, a medical info extraction stage 444, an entity linking stage 446, and a relationship extractions stage 448. Additional structured medical data is produced and provided to an additional structured medical data database 450.

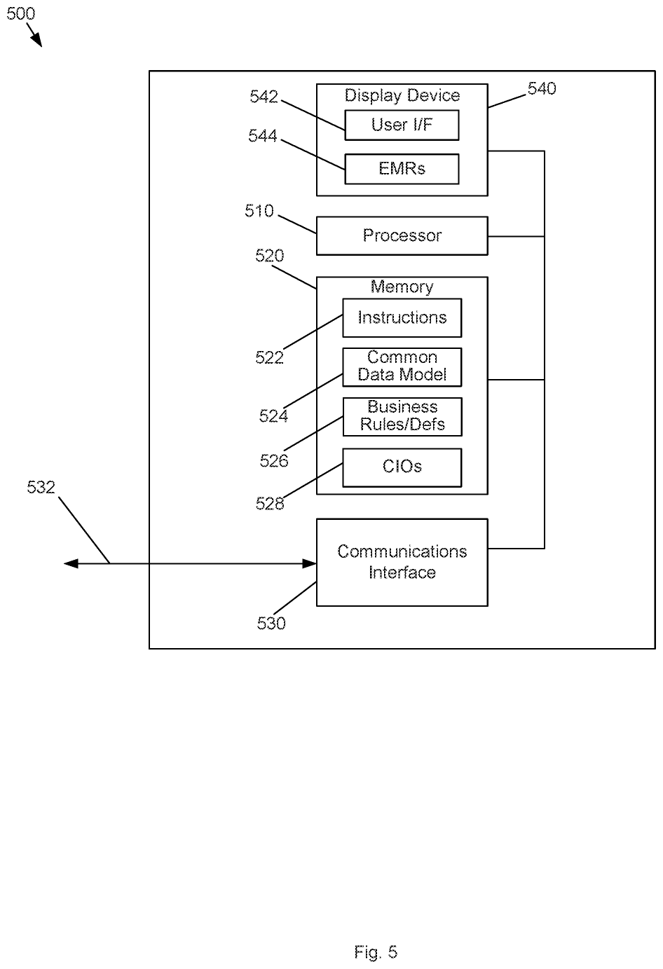

FIG. 5 is a device 500 used in the payer-provider collaboration system.

In FIG. 5, the device may be a device used on the healthcare provider side, a device used on the health insurer side, or a device that is used at the payer-provider collaboration system. In FIG. 5, the device includes a processor 510, memory 520, communication interface 530, and display device 540. Memory 520 may store instructions 522 for implementing the payer-provider collaboration system as described herein. Communication interface 530 processes data transferred via path 532 between the healthcare provider side and the payer-provider collaboration system, and/or data transferred between the health insurer side and the payer-provider collaboration system.

The processor 510 may execute the instructions 522 to receive data from one or more healthcare providers and claims data provided by one or more health insurers, and create a common data model 524 using the data from one or more healthcare providers and claims data provided by one or more health insurers. The processor 510 receive business rules and definitions of opportunities of interest 526 from one or more health insurers. Data from the common data model 524 and the business rules and definitions of opportunities of interest are processed by the processor to identify one or more care improvement opportunities (CIOs) 528 to address.

Stakeholder specific user interfaces 542 may be generated based on data from the common data model 528, and results from analytics performed by the processor 510. User interface 542 are used by authenticated health plan users, provider users, and operational staff to interact with the data, and act on identified CIOs 528. The user interface 542 may include health plan user interfaces, provider group user interfaces, and system operator interfaces 142. For example, the user interface 542 may be a first user interface presented on a display device 540 at one or more healthcare providers. The user interface 542 may present one or more CIOs to the one or more healthcare providers so that the one or more healthcare providers can address the CIOs 528. The user interface 542 presented on a display device 540 at one or more healthcare providers may be used to identify, from the one or more CIOs 528, actions to take by the one or more healthcare providers to improve care and drive completion of intervention across the one or more healthcare providers. The user interface 542 presented on a display device 540 at one or more healthcare providers may further be used to identify of gaps in healthcare actions and identification of additional services to provide a patient, to identify of gaps in coding and documentation of the one or more CIOs 528, to identify social determinants of health (SDoH) and activities of daily living (ADL) limitations to mitigate, to identify interventions for care not being implemented, to identify failure to follow through with select elements of a treatment plan, and to identify an impact of the one or more CIOs 528 on operational, clinical, and financial metrics.

By way of further example, the user interface 542 may be a second user interface presented on a display device 540 at one or more health insurers. The user interface 542 may present to the one or more health insurers data about opportunities addressed by the one or more healthcare providers, and data identifying incentive payments to pay to the one or more healthcare providers based on the data about opportunities addressed by the one or more healthcare providers. The user interface 542 presented on the display device 540 at the one or more health insurers may be used to address a systemic issue. A health insurer becomes aware of a systemic failure in the delivery of key elements of care and/or a failure to fully document clinical encounters. The user interface 542 presented on the display device 540 at the one or more health insurers may be used to define a systemic issue to address, including: (i) naming the health issue to address, (ii) assigning it three “priority” scores, clinical impact, financial impact, and ease-to-address, (iii) a target time frame for each clinician to address the issue, (iv) incentive points associated with timely and successful completion of the corrective action, and (v) target distribution of the incentives points among all members of a patient's care team that address the issue. The user interface 542 on display device 540 at the one or more health insurers may further present data about opportunities addressed by the one or more healthcare providers and identify incentive payments to pay to the one or more healthcare providers

The CIOs 528 presented in the user interface 540 on display device 540 may include a health issue to address, a priority scores based on a clinical impact score, a financial impact score, an ease-to-address score, a target time frame for the one or more healthcare providers to address the health issue, identification of incentive associated with timely and successful completion of the corrective action, and rules controlling target distribution of the incentives among all members of the one or more healthcare providers that address the issue.

The user interface 542 may present additional interfaces to the healthcare providers or health insurers. For example, the user interface 542 may be a third user interface that is presented on the display device 540 at one or more healthcare providers to display the one or more CIOs 528 and to document one or more actions taken to address the one or more CIOs 528. The user interface 542 may also be implemented as a fourth user interface that is presented on the display device 540 at the one or more health insurers to execute searches to determine one or more CIOs 528. The user interface 542 may also be implemented as a fifth user interface that is presented on the display device 540 at the one or more health insurers to identify one or more actions taken by the healthcare providers regarding the CIOs 528, to verify the one or more actions taken regarding the CIOs 528, and to execute payment of incentives to the one or more healthcare providers in response to the verification of the one or more actions taken regarding the CIOs by the one or more healthcare providers

As described above with regard to FIG. 1, the user interface 542 may include a filter control to filter the CIOs 528 by type, urgency, priority based on clinical priority, financial priority, and likely ease to address, and eligible incentives, and to push the one or more CIOs 528 to a care team associated with each patient in a target cohort. CIOs 528 may also be published in one or more EMRs 544.

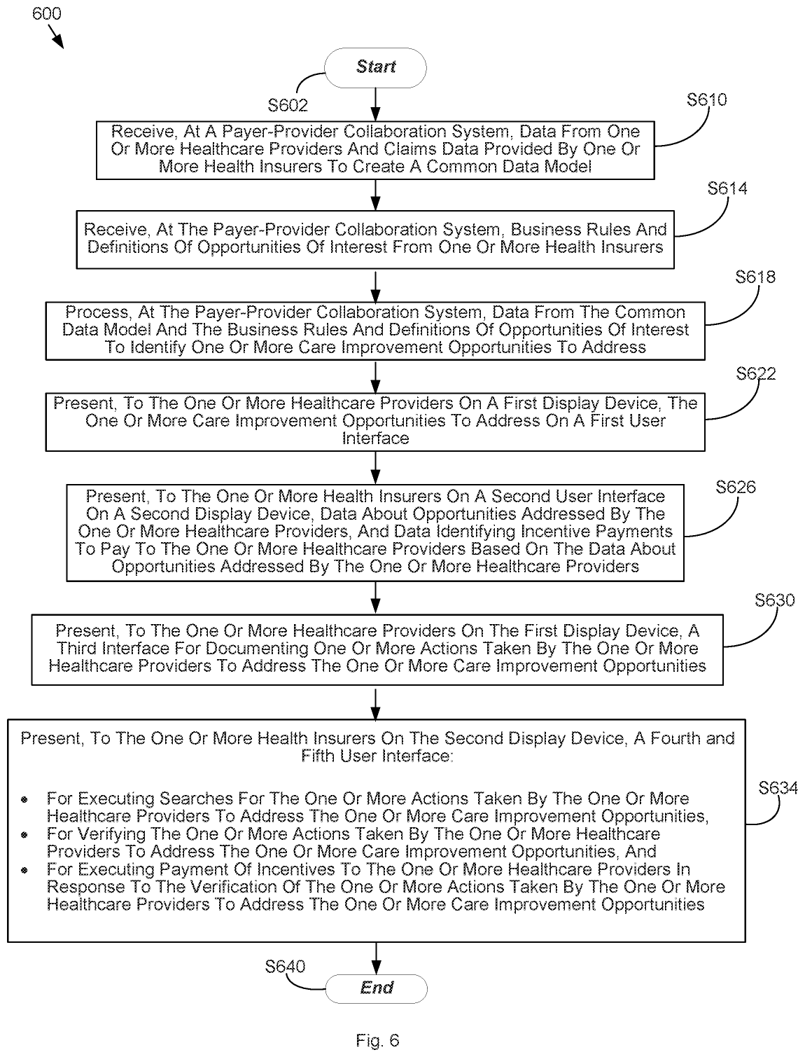

FIG. 6 is a flow chart of a method 600 for providing a system for healthcare collaboration between healthcare providers and health insurers.

In FIG. 6, method 600 starts (S502), and a payer-provider collaboration system receives data from one or more healthcare providers and claims data provided by one or more health insurers to create a common data model (S610). Referring to FIG. 1, a payer-provider collaboration system 120 is coupled to healthcare providers 150 and to health insurers 170. Structured data 158 and unstructured data 160 is provided to the payer-provider collaboration system 120 from the healthcare providers 150. Health insurers 170 may include one or more health plan claims systems 172, 174. The health plan claims systems 172, 174 provide claims data 176 to the payer-provider collaboration system 120.

The payer-provider collaboration system receives business rules and definitions of opportunities of interest from one or more health insurers (S614). Referring to FIG. 1, one or more health plan clinical leadership teams 178 create and modify as necessary business rules and definitions of care improvement opportunities 130, which are also provided to the payer-provider collaboration system 120. These may include clinical definitions, priorities, incentives for providers to address, and timing parameters 132.

The payer-provider collaboration system processes data from the common data model and the business rules and definitions of opportunities of interest to identify one or more care improvement opportunities to address (S618). Referring to FIG. 1, analytics 128 are used to process data 126 from the common data model 122 and the business rules and definitions of opportunities of interest 130 to identify one or more care improvement opportunities to address 134.

The one or more care improvement opportunities to address are presented to the one or more healthcare providers on a first user interface on a first display device (S622). Referring to FIG. 1, patient care improvement opportunities to address 144 may be presented from the care improvement opportunities database 138 to the healthcare providers 150. For example, the one or more care improvement opportunities 144 from the care improvement opportunities database 138 may be presented in a first of the stakeholder specific user interfaces 140 on a display device to the one or more healthcare providers150.

Data about opportunities addressed by the one or more healthcare providers, and data identifying incentive payments to pay to the one or more healthcare providers based on the data about opportunities addressed by the one or more healthcare providers are presented to the one or more health insurers on a second user interface on a second display device (S626). Referring to FIG. 1, the care improvement opportunities database 138 may also identify to the health insurers 170 incentive information 148 that includes improvement opportunities that were address and incentive payments that are due to the healthcare providers 170. For example, data about opportunities addressed by the one or more healthcare providers and identifying incentive payments to pay to the one or more healthcare providers 148 may be presented in a second of the stakeholder specific user interfaces 140 on a display device to the health insurers 170.

A third interface is presented to the one or more healthcare providers on the first display device for documenting one or more actions taken by the one or more healthcare providers to address the one or more care improvement opportunities (S630). Referring to FIG. 1, a third interface at the one or more healthcare providers 150 to present the one or more of the care improvement opportunities 138 and to document one or more actions taken to address the one or more care improvement opportunities 138.

A fourth and fifth user interface are presented to the one or more health insurers on the second display device for executing searches for the one or more actions taken by the one or more healthcare providers to address the one or more care improvement opportunities, for verifying the one or more actions taken by the one or more healthcare providers to address the one or more care improvement opportunities, and for executing payment of incentives to the one or more healthcare providers in response to the verification of the one or more actions taken by the one or more healthcare providers to address the one or more care improvement opportunities (S634). Referring to FIG. 1, a fourth user interface may be used to presented to the one or more health insurers 170 to use for executing searches to determine one or more care improvement opportunities 138. A fifth user interface may be used to identify one or more actions taken by the healthcare providers 150 regarding the care improvement opportunities 138, to verify the one or more actions taken regarding the care improvement opportunities 138, and to execute payment of incentives to the healthcare providers 150 in response to the verification of the one or more actions taken regarding the care improvement opportunities 138.

The method then ends (S640).

The subject matter of the present disclosure may be provided as a computer program product including one or more non-transitory computer-readable storage media having stored thereon instructions (in compressed or uncompressed form) that may be used to program a computer (or other electronic device) to perform processes or methods described herein. The computer-readable storage media may include one or more of an electronic storage medium, a magnetic storage medium, an optical storage medium, a quantum storage medium, or the like. For example, the computer-readable storage media may include, but are not limited to, hard drives, floppy diskettes, optical disks, read-only memories (ROMs), random access memories (RAMs), erasable programmable ROMs (EPROMs), electrically erasable programmable ROMs (EEPROMs), flash memory, magnetic or optical cards, solid-state memory devices, or other types of physical media suitable for storing electronic instructions.

Further, the subject matter of the present disclosure may also be provided as a computer program product including a transitory machine-readable signal (in compressed or uncompressed form). Examples of machine-readable signals, whether modulated using a carrier or unmodulated, include, but are not limited to, signals that a computer system or machine hosting or running a computer program may be configured to access, including signals transferred by one or more networks. For example, a transitory machine-readable signal may comprise transmission of software by the Internet.

Separate instances of these programs can be executed on or distributed across any number of separate computer systems. Thus, although certain steps have been described as being performed by certain devices, software programs, processes, or entities, this need not be the case. A variety of alternative implementations will be understood by those having ordinary skill in the art.

Additionally, those having ordinary skill in the art readily recognize that the techniques described above can be utilized in a variety of devices, environments, and situations. Although the subject matter has been described in language specific to structural features or methodological acts, it is to be understood that the subject matter defined in the appended claims is not necessarily limited to the specific features or acts described. Rather, the specific features and acts are disclosed as exemplary forms of implementing the claims.

What is claimed is:

1. A payer-provider collaboration system, comprising:
a memory storing computer-readable instructions; and
a processor, coupled to the memory, configured to execute the computer-readable instructions to:
receive data from one or more healthcare providers and claims data provided by one or more health insurers to create a common data model;
receive business rules and definitions of opportunities of interest from one or more health insurers;
process data from the common data model and the business rules and definitions of opportunities of interest to identify one or more care improvement opportunities to address; and
present, in a first user interface on a first display device, the one or more care improvement opportunities to the one or more healthcare providers, and present to the one or more health insurers in a second user interface on a second display device, data about opportunities addressed by the one or more healthcare providers, and data identifying incentive payments to pay to the one or more healthcare providers based on the data about opportunities addressed by the one or more healthcare providers.

2. The payer-provider collaboration system of claim 1, wherein the one or more care improvement opportunities identify actions to take to improve care and drive completion of intervention across the one or more healthcare providers.

3. The payer-provider collaboration system of claim 1, wherein the common data model is created by the processor, the processor further configured to:
receive structured and unstructured data from the one or more healthcare providers;
process the unstructured data through a natural language processing (NLP) pipeline to create additional structured data;
receive the claims data from one or more health plan claims systems of the one or more health insurers; and
create the common data model using the structured data, the additional structured data, and the claims data from the one or more health plan claims systems.

4. The payer-provider collaboration system of claim 1, wherein the one or more care improvement opportunities are presented in an electronic medical record (EMR).

5. The payer-provider collaboration system of claim 1, wherein the business rules and definitions of opportunities of interest include clinical definitions, and priorities and healthcare incentives for addressing management of care opportunities by the one or more healthcare providers.

6. The payer-provider collaboration system of claim 1, wherein the processor is further configured to present a third interface, to the one or more healthcare providers, to present the one or more care improvement opportunities, and to document one or more actions taken to address the one or more care improvement opportunities.

7. The payer-provider collaboration system of claim 6, wherein the processor is further configured to present, to the one or more health insurers:
a fourth user interface for executing searches to determine one or more care improvement opportunities; and
a fifth user interface to identify, from the one or more care improvement opportunities, the one or more actions taken by the one or more healthcare providers regarding the one or more care improvement opportunities, to verify the one or more actions taken regarding the one or more care improvement opportunities, and to execute payment of incentives to the one or more healthcare providers in response to the verification of the one or more actions taken regarding the one or more care improvement opportunities, wherein the incentives are tied to database for translation to monetary compensation values and rules.

8. The payer-provider collaboration system of claim 6, wherein the processor is further configured to determine the one or more care improvement opportunities by analyzing medical records, wherein the one or more care improvement opportunities include coding actions, quality and care improvement interventions, revenue enhancing interventions based on a review of patient medical records and at least one patient profile.

9. The payer-provider collaboration system of claim 8, wherein the at least one patient profile includes at least a patient name, data regarding drug exposures and health condition occurrences.

10. The payer-provider collaboration system of claim 1, wherein the one or more care improvement opportunities to address include one or more of identification of gaps in healthcare actions and identification of additional services to provide a patient, identification of gaps in coding and documentation of the one or more care improvement opportunities, identification of social determinants of health (SDoH) and activities of daily living (ADL) limitations to mitigate, identification of interventions for care not being implemented, identification of failure to follow through with select elements of a treatment plan, and identification of an impact of the one or more care improvement opportunities on operational, clinical, and financial metrics.

11. The payer-provider collaboration system of claim 1, wherein the claims data provided by one or more health insurers include member identifiers, date of service, medical procedure and service codes, diagnosis coded, point of service code, and other medical claim elements.

12. The payer-provider collaboration system of claim 1, wherein the processor is further configured to processes the data from the common data model and the business rules and definitions of opportunities of interest to identify the one or more care improvement opportunities to address by performing analytics on the common data model and the business rules and definitions of opportunities of interest.

13. The payer-provider collaboration system of claim 1, wherein the data from one or more healthcare providers and from claims data provided by one or more health insurers are provided to create the common data model using standardized data exchange protocols that define inputs and outputs of interoperable clinical data associated with patients, clinicians, and automated data feeds.

14. The payer-provider collaboration system of claim 1, wherein the processor is further configured to identify the one or more care improvement opportunities to address by matching a patient identifier from the one or more healthcare providers with a patient member identifier from the one or more health insurers.

15. The payer-provider collaboration system of claim 1, wherein the processor is further configured to present, in the at least one of the first user interface and the second user interface, the care improvement opportunities including a health issue to address, a priority scores based on a clinical impact score, a financial impact score, an ease-to-address score, a target time frame for the one or more healthcare providers to address the health issue, identification of incentive associated with timely and successful completion of corrective action, and rules controlling target distribution of the incentives among all members of the one or more healthcare providers that address the issue.

16. The payer-provider collaboration system of claim 1, wherein the processor is further configured to filter the one or more care improvement opportunities by type, urgency, priority based on clinical priority, financial priority, and likely ease to address, and eligible incentives, and to push the one or more care improvement opportunities to a care team associated with each patient in a target cohort.

17. The payer-provider collaboration system of claim 1, wherein the common data model is configured to provide physical, network, and process security measures for protected health information.

18. A method for providing payer-provider collaboration, comprising:
receiving data from one or more healthcare providers and claims data provided by one or more health insurers to create a common data model;
receiving business rules and definitions of opportunities of interest from one or more health insurers;
processing data from the common data model and the business rules and definitions of opportunities of interest to identify one or more care improvement opportunities to address; and
presenting, in a first user interface on a first display device, the one or more care improvement opportunities to the one or more healthcare providers to address, and presenting to the one or more health insurers in a second user interface on a second display device, data about opportunities addressed by the one or more healthcare providers, and data identifying incentive payments to pay to the one or more healthcare providers based on the data about opportunities addressed by the one or more healthcare providers.

19. The method of claim 18, further comprising identifying, from the one or more care improvement opportunities, actions to take to improve care and drive completion of intervention across the one or more healthcare providers.

20. The method of claim 18, wherein the creating the common data model further comprises:
receiving structured and unstructured data from the one or more healthcare providers;
processing the unstructured data through a natural language processing (NLP) pipeline to create additional structured data;
receiving the claims data from one or more health plan claims systems of the one or more health insurers; and
creating the common data model using the structured data, the additional structured data, and the claims data from the one or more health plan claims systems.

21. The method of claim 18 further comprising presenting the one or more care improvement opportunities in an electronic medical record (EMR).

22. The method of claim 18, wherein the processing data from the business rules and definitions of opportunities of interest to identify one or more care improvement opportunities to address further comprises identifying the one or more care improvement opportunities using clinical definitions, and priorities and healthcare incentives for addressing management of care opportunities by the one or more healthcare providers.

23. The method of claim 18, further comprising presenting a third interface, to the one or more healthcare providers, to present the one or more care improvement opportunities, and to document one or more actions taken to address the one or more care improvement opportunities.

24. The method of claim 23, further comprises presenting, to the one or more health insurers:
a fourth user interface for executing searches to determine one or more care improvement opportunities; and
a fifth user interface to identify, from the one or more care improvement opportunities, the one or more actions taken by the one or more healthcare providers regarding the one or more care improvement opportunities, to verify the one or more actions taken regarding the one or more care improvement opportunities, and to execute payment of incentives to the one or more healthcare providers in response to the verification of the one or more actions taken regarding the one or more care improvement opportunities, wherein the incentives are tied to database for translation to monetary compensation values and rules.

25. The method of claim 23, further comprising determining the one or more care improvement opportunities by analyzing medical records, wherein the determining the one or more care improvement opportunities includes determining coding actions, quality and care improvement interventions, revenue enhancing interventions based on a review of patient medical records and at least one patient profile.

26. The method of claim 25, wherein the analyzing the revenue enhancing interventions based on the review of the patient medical records and the at least one patient profile further comprises analyzing at least a patient name, data regarding drug exposures and health condition occurrences.

27. The method of claim 18, wherein the presenting, in the first user interface on the first display device, the one or more care improvement opportunities to the one or more healthcare providers to address further comprises one or more of identification of gaps in healthcare actions and identification of additional services to provide a patient, identification of gaps in coding and documentation of the one or more care improvement opportunities, identification of social determinants of health (SDoH) and activities of daily living (ADL) limitations to mitigate, identification of interventions for care not being implemented, identification of failure to follow through with select elements of a treatment plan, and identification of an impact of the one or more care improvement opportunities on operational, clinical, and financial metrics.

28. The method of claim 18, wherein the receiving claims data provided by one or more health insurers further comprises receiving member identifiers, date of service, medical procedure and service codes, diagnosis coded, point of service code, and other medical claim elements.

29. The method of claim 18, wherein the processing data from the common data model and the business rules and definitions of opportunities of interest further comprises performing analytics on the common data model and the business rules and definitions of opportunities of interest.

30. The method of claim 18, wherein the receiving the data from the one or more healthcare providers and the claims data provided by one or more health insurers to create the common data model further comprises using standardized data exchange protocols that define inputs and outputs of interoperable clinical data associated with patients, clinicians, and automated data feeds.

31. The method of claim 18, wherein the identifying the one or more care improvement opportunities to address further comprises matching a patient identifier from the one or more healthcare providers with a patient member identifier from the one or more health insurers.

32. The method of claim 18, further comprise presenting, in the at least one of the first user interface and the second user interface, the care improvement opportunities including a health issue to address, a priority scores based on a clinical impact score, a financial impact score, an ease-to-address score, a target time frame for the one or more healthcare providers to address the health issue, identification of incentive associated with timely and successful completion of corrective action, and rules controlling target distribution of the incentives among all members of the one or more healthcare providers that address the issue.

33. The method of claim 18, further comprising filtering the one or more care improvement opportunities by type, urgency, priority based on clinical priority, financial priority, and likely ease to address, and eligible incentives, and pushing the one or more care improvement opportunities to a care team associated with each patient in a target cohort.

34. The method of claim 18, wherein the creating the common data model further comprises configuring the common data model with physical, network, and process security measures for protected health information.

35. A non-transitory computer-readable media having computer-readable instructions stored thereon, which when executed by a processor causes the processor to perform operations to provide a payer-provider collaboration, the operations comprising:
receiving data from one or more healthcare providers and claims data provided by one or more health insurers to create a common data model;
receiving business rules and definitions of opportunities of interest from one or more health insurers;
processing data from the common data model and the business rules and definitions of opportunities of interest to identify one or more care improvement opportunities to address; and
presenting, in a first user interface on a first display device, the one or more care improvement opportunities to the one or more healthcare providers to address, and presenting to the one or more health insurers in a second user interface on a second display device, data about opportunities addressed by the one or more healthcare providers, and data identifying incentive payments to pay to the one or more healthcare providers based on the data about opportunities addressed by the one or more healthcare providers.

36. The non-transitory computer-readable media of claim 35, further comprising identifying, from the one or more care improvement opportunities, actions to take to improve care and drive completion of intervention across the one or more healthcare providers.

37. The non-transitory computer-readable media of claim 35, wherein the creating the common data model further comprises:
receiving structured and unstructured data from the one or more healthcare providers;
processing the unstructured data through a natural language processing (NLP) pipeline to create additional structured data;
receiving the claims data from one or more health plan claims systems of the one or more health insurers; and
creating the common data model using the structured data, the additional structured data, and the claims data from the one or more health plan claims systems.

38. The non-transitory computer-readable media of claim 35 further comprising presenting the one or more care improvement opportunities in an electronic medical record (EMR).

39. The non-transitory computer-readable media of claim 35, wherein the processing data from the business rules and definitions of opportunities of interest to identify one or more care improvement opportunities to address further comprises identifying the one or more care improvement opportunities using clinical definitions, and priorities and healthcare incentives for addressing management of care opportunities by the one or more healthcare providers.

40. The non-transitory computer-readable media of claim 35, further comprising presenting a third interface, to the one or more healthcare providers, to present the one or more care improvement opportunities, and to document one or more actions taken to address the one or more care improvement opportunities.

41. The non-transitory computer-readable media of claim 40, further comprises presenting, to the one or more health insurers:
a fourth user interface for executing searches to determine one or more care improvement opportunities; and
a fifth user interface to identify, from the one or more care improvement opportunities, the one or more actions taken by the one or more healthcare providers regarding the one or more care improvement opportunities, to verify the one or more actions taken regarding the one or more care improvement opportunities, and to execute payment of incentives to the one or more healthcare providers in response to the verification of the one or more actions taken regarding the one or more care improvement opportunities, wherein the incentives are tied to database for translation to monetary compensation values and rules.

42. The non-transitory computer-readable media of claim 40, further comprising determining the one or more care improvement opportunities by analyzing medical records, wherein the determining the one or more care improvement opportunities includes determining coding actions, quality and care improvement interventions, revenue enhancing interventions based on the review of patient medical records and the at least one patient profile.

43. The non-transitory computer-readable media of claim 42, wherein the analyzing the revenue enhancing interventions based on a review of patient medical records and at least one patient profile further comprises analyzing at least a patient name, data regarding drug exposures and health condition occurrences.

44. The non-transitory computer-readable media of claim 35, wherein the presenting, in the first user interface on the first display device, the one or more care improvement opportunities to the one or more healthcare providers to address further comprises one or more of identification of gaps in healthcare actions and identification of additional services to provide a patient, identification of gaps in coding and documentation of the one or more care improvement opportunities, identification of social determinants of health (SDoH) and activities of daily living (ADL) limitations to mitigate, identification of interventions for care not being implemented, identification of failure to follow through with select elements of a treatment plan, and identification of an impact of the one or more care improvement opportunities on operational, clinical, and financial metrics.

45. The non-transitory computer-readable media of claim 35, wherein the receiving claims data provided by one or more health insurers further comprises receiving member identifiers, date of service, medical procedure and service codes, diagnosis coded, point of service code, and other medical claim elements.

46. The non-transitory computer-readable media of claim 35, wherein the processing data from the common data model and the business rules and definitions of opportunities of interest further comprises performing analytics on the common data model and the business rules and definitions of opportunities of interest.

47. The non-transitory computer-readable media of claim 35, wherein the receiving the data from the one or more healthcare providers and the claims data provided by one or more health insurers to create the common data model further comprises using standardized data exchange protocols that define inputs and outputs of interoperable clinical data associated with patients, clinicians, and automated data feeds.

48. The non-transitory computer-readable media of claim 35, wherein the identifying the one or more care improvement opportunities to address further comprises matching a patient identifier from the one or more healthcare providers with a patient member identifier from the one or more health insurers.

49. The non-transitory computer-readable media of claim 35, further comprise presenting, in the at least one of the first user interface and the second user interface, the care improvement opportunities including a health issue to address, a priority scores based on a clinical impact score, a financial impact score, an ease-to-address score, a target time frame for the one or more healthcare providers to address the health issue, identification of incentive associated with timely and successful completion of corrective action, and rules controlling target distribution of the incentives among all members of the one or more healthcare providers that address the issue.

50. The non-transitory computer-readable media of claim 35, further comprising filtering the one or more care improvement opportunities by type, urgency, priority based on clinical priority, financial priority, and likely ease to address, and eligible incentives, and pushing the one or more care improvement opportunities to a care team associated with each patient in a target cohort.

51. The non-transitory computer-readable media of claim 35, wherein the creating the common data model further comprises configuring the common data model with physical, network, and process security measures for protected health information.
